(19) United States
(12) Patent Application Publication
(10) Pub. No.: US 2024/0398606 A1
(43) Pub. Date: Dec. 5, 2024

(54) METHOD FOR SCANNING STOMA AND PERISTOMAL TOPOGRAPHY AND GENERATING REPRESENTATIONS OF SAME

(71) Applicant: Hollister Incorporated, Libertyville, IL (US)

(72) Inventors: Gregory J. Czaplewski, Bloomingdale, IL (US); Mark W. Jockel, Vernon Hills, IL (US)

(21) Appl. No.: 18/698,119

(22) PCT Filed: Oct. 26, 2022
(86) PCT No.: PCT/US2022/047879

§ 371 Date: Apr. 3, 2024

Related U.S. Application Data
(60) Provisional application No. 63/272,349, filed on Oct. 27, 2021.

(51) Int. Cl. A61F 5/445; A61B 5/107; B33Y 50/00

(52) U.S. Cl. CPC A61F 5/445; A61B 5/1077; A61B 5/1079; B33Y 50/00

(57) ABSTRACT

A method of scanning stoma and peristomal topography and generating representations thereof. The method can comprise taking a three-dimensional scan of a person's stoma and peristomal areas to generate a set of data points in space. The data points can correspond to points along physical external surfaces of the stomal and peristomal areas. The set of data points can be mapped into a three-dimensional point cloud. The data points of the point cloud can reflect relative sizes, shapes, convexity characteristics, and topographies of features along external surfaces of the stoma and peristomal areas. The point cloud can be converted into a digital three-dimensional solid body model. A physical three-dimensional representation can be created which can be representative of the person's stoma and peristomal areas. The physical three-dimensional representation can be used for evaluating concepts for implementation in ostomy appliances.

### CROSS-REFERENCE TO RELATED APPLICATION

This application is based upon and claims priority to Provisional Application No. 63/272,349 filed on Oct. 27, 2021, the entire contents thereof are incorporated herein by reference in their entireties for all purposes.

### BACKGROUND

The present disclosure relates to the development of ostomy appliances, and more particularly to the collection of data on the shape and topography of a stoma and peristomal area to generate a representation of same for use in designing, evaluating and fabricating ostomy appliances such as skin barriers and stoma support rings.

Ostomy appliances for the collection of body waste output from a stoma are well known. Generally, ostomy appliances can include a pouch that can be attached to a user via an ostomy barrier, which is configured to seal against peristomal skin surfaces and protect the peristomal surfaces from exposure to stomal effluent. However, the topography of stomas and peristomal surfaces surrounding stomas can vary among patients and providing a single ostomy appliance which can effectively seal against such different peristomal surfaces and stomas can be particularly challenging. For example, stomas may have different circumferences and/or undulating or uneven peristomal surfaces.

Devices such as template sheets, adhesive wafers and implantable skin grafts in various forms are generally known and used to fabricate a skin barrier to attempt to better correspond to the shape and size of individual stomas. Such devices and solutions, however, are either invasive, difficult or time-consuming to create or use and/or not precise from the standpoint of generating a customized skin barrier that conforms to the contours of a particular user's stomal region or common stomal topographies.

Thus, there is a critical need in the art for systems and methods for collecting data regarding the topographies of stoma and peristomal areas for the purpose of fabricating improved ostomy appliances that are better tailored to particular individuals or groups of individuals having certain stomal characteristics. For example, it would be particularly advantageous to be able to collect and use such data to assess the performance of certain ostomy design concepts to eliminate the need for costly clinical evaluations and/or to guide clinicians in understanding how “adaptable” a particular ostomy product may be based on its ability to fit common stomal and peristomal topographies. It would be further advantageous to be able to collect and utilize such data to guide the product selection of an end user.

Three-dimensional (“3D”) scanning, modeling and printing technologies are generally known and have been shown to transform traditional production capabilities by economizing product design and facilitating personal fabrication and quick turn-around times. From the subject disclosure, persons of ordinary skill in the art will recognize and appreciate that it would be advantageous to utilize certain 3D scanning, modeling and printing capabilities to help with the fabrication of improved ostomy appliances that are better tailored to particular individuals or groups of individuals having certain stomal characteristics.

### BRIEF SUMMARY

Embodiments presented herein are directed to a method comprising the scanning of stoma and peristomal topography and generating representations of same. The method can comprise taking a three-dimensional scan of a person's stoma and peristomal areas. The scan can generate a set of data points in space which correspond to points along physical external surfaces of the stomal and peristomal areas. The set of data points can be mapped into a three-dimensional point cloud. The data points of the point cloud can reflect relative sizes, shapes, convexity characteristics, and topographies of features along external surfaces of the stoma and peristomal areas. The point cloud can be converted into a digital three-dimensional solid body model being visually representative of the person's stoma and peristomal areas. A physical three-dimensional representation of the digital solid body model can be created which corresponds to the person's stoma and peristomal areas. The physical three-dimensional representation can be used for evaluating concepts for implementation in ostomy appliances.

According to example embodiments, a three-dimensional visual light scanner can be provided for the taking of the three-dimensional scan of a person's stoma and peristomal areas. Digital surface reconstruction can be utilized for converting the point cloud into a digital three-dimensional solid body model. The physical three-dimensional representation can be created by a three-dimensional printer or by molding processes such as injection molding or cast molding through the use of a mold filled with a pliable mold material. As part of the evaluation, a prototype embodying the concept can be applied onto at least a portion of the physical three-dimensional representation. The topographies of features along the external surfaces of the person's stoma and peristomal areas can be reproduced on the physical three-dimensional representation.

Embodiments presented herein are further directed to a method whereby a three-dimensional scan of a person's stoma and peristomal areas can be taken. Such scan can generate a set of data points in space which correspond to points along physical external surfaces of the stomal and peristomal areas. The set of data points can be mapped into a three-dimensional point cloud. The data points of the point cloud can reflect relative sizes, shapes and topographies of features along external surfaces of the stoma and peristomal areas. The point cloud can be converted into a digital three-dimensional solid body model that is visually representative of the person's stoma and peristomal areas. The set of data points can be electronically stored with data points from scans of other persons' stoma and peristomal areas to identify stomal characteristics that are prevalent within a population. An ostomy appliance can be provided to accommodate a stomal region having the characteristics.

According to example embodiments, a three-dimensional scanner such as a visible light scanner, a scanner which utilizes wavelengths of non-visible light such as infrared, or other scanning devices such as magnetic resonance imaging (MRI) or LIDAR (light detection and ranging) can be used to take the three-dimensional scan of a person's stoma and peristomal areas. Digital surface reconstruction can be utilized for converting the point cloud into a digital three-dimensional solid body model. The stomal characteristics can be made selectable via a user interface. A digital rendering of the ostomy appliance can be visually presented with image data captured by a digital camera, the image data showing a user's stomal region. The ostomy appliance can comprise at least one of an ostomy skin barrier and a stoma support ring. The ostomy appliance can comprise a convex insert with a convexity depth. The ostomy appliance can comprise a convex insert with a tension location. The ostomy appliance can comprise a convex insert with a convexity slope.

Embodiments presented herein are further directed to a method whereby a three-dimensional scan of a person's stoma and peristomal areas can be taken and a set of data points in space can be generated which correspond to points along physical external surfaces of the stomal and peristomal areas. The set of data points can be mapped into a three-dimensional point cloud. The data points of the point cloud can reflect relative sizes, shapes and topographies of features along external surfaces of the stoma and peristomal areas. The point cloud can be converted into a digital three-dimensional solid body model being visually representative of the person's stoma and peristomal areas. The set of data points can be electronically stored with data points from scans of other persons' stoma and peristomal areas to identify stomal characteristics that are prevalent within a population. The stomal characteristics can be made selectable via a user interface and correspond to an ostomy appliance designed to accommodate the characteristics.

According to example embodiments, a digital rendering of the ostomy appliance can be visually presented with image data captured by a digital camera, the image data showing a user's stomal region. An ostomy appliance can be provided to accommodate a stomal region having the characteristics. The ostomy appliance can comprise at least one of an ostomy skin barrier and a stoma support ring.

Other objects, advantages and features of the present disclosure will be understood and appreciated by persons of ordinary skill in the art from consideration of the following specification taken in conjunction with the accompanying drawings.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a flow diagram of a method for generating a representation of a stoma and peristomal topography, according to an embodiment.

FIG. 2A is an illustration of an ostomy system, according to an embodiment.

FIG. 2B is an illustration of the ostomy system of FIG. 2A attached to a user.

FIG. 3 is an illustration of a depth of a convex skin barrier.

FIG. 4 is an illustration of the compressibility of a convex skin barrier.

FIG. 5 is an illustration of the flexibility of a convex skin barrier.

FIGS. 6A and 6B are illustrations of tension locations of a convex skin barrier.

FIG. 7 is an illustration of a slope of a convex skin barrier.

FIG. 8 is a cross-section illustration of a user's abdomen with a stoma, according to an embodiment.

FIG. 9 is an annotated cross-section illustration of the user's abdomen of FIG. 8.

FIG. 10 is a cross-section illustration of a convex insert, according to an embodiment.

FIG. 11 is the cross-section illustration of the user's abdomen of FIG. 8 with the convex insert of FIG. 10.

FIG. 12 is a cross-section illustration of the convex insert of FIG. 10.

FIG. 13 is a flow diagram illustrating a method for mapping a stoma and peristomal area in an image, according to an embodiment.

FIG. 14 is a schematic illustration of a computing environment, according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,” “down,” front,” “back,” and the like are used for purposes of illustration and as such, are not limiting. Depending on the context, the word “if” as used herein may be interpreted as “when” or “upon” or “in response to determining.”

The present disclosure provides methods for generating a digital representation of a scanned stoma and peristomal topography. The digital representation can be used to better mount an ostomy system around a stoma based on the characteristics of convexity of a skin barrier. The ostomy system can include an ostomy barrier of a one-piece pouch system or a faceplate for a two-piece pouch system.

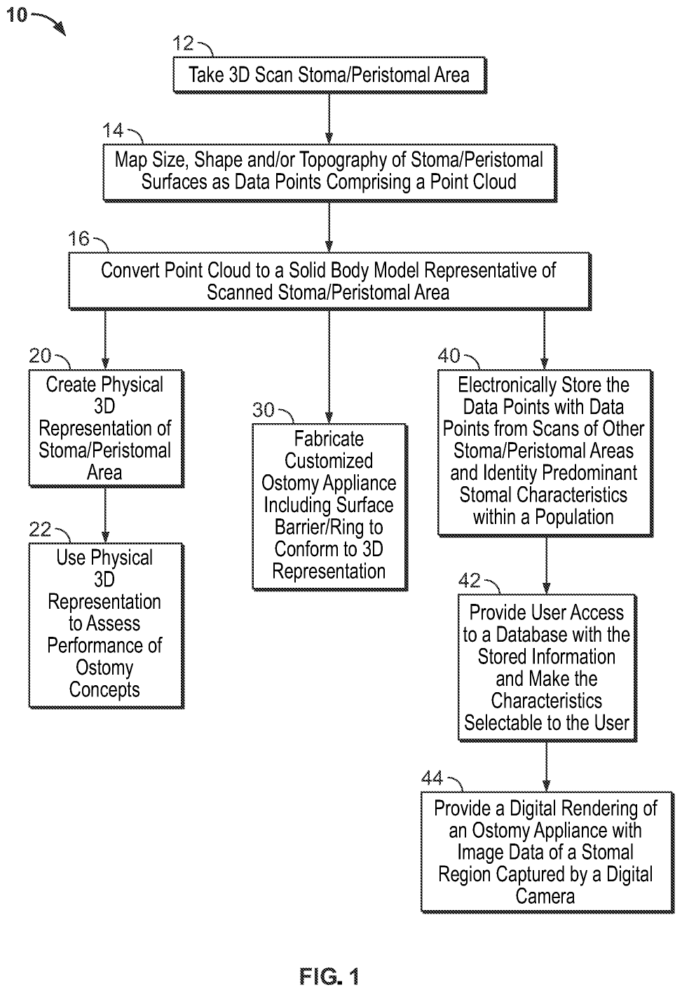

FIG. 1 shows method 10 according to example embodiments presented herein. As shown schematically in FIG. 1, method 10 can include taking a 3D scan of a stoma and peristomal area, mapping the stoma and peristomal area, converting the mapped stoma and peristomal area into the digital representation, and either 3D printing the digital representation, fabricating an ostomy appliance, such as a convex skin barrier or convex insert (FIG. 10), based on the digital representation, or storing the digital representation for further processing.

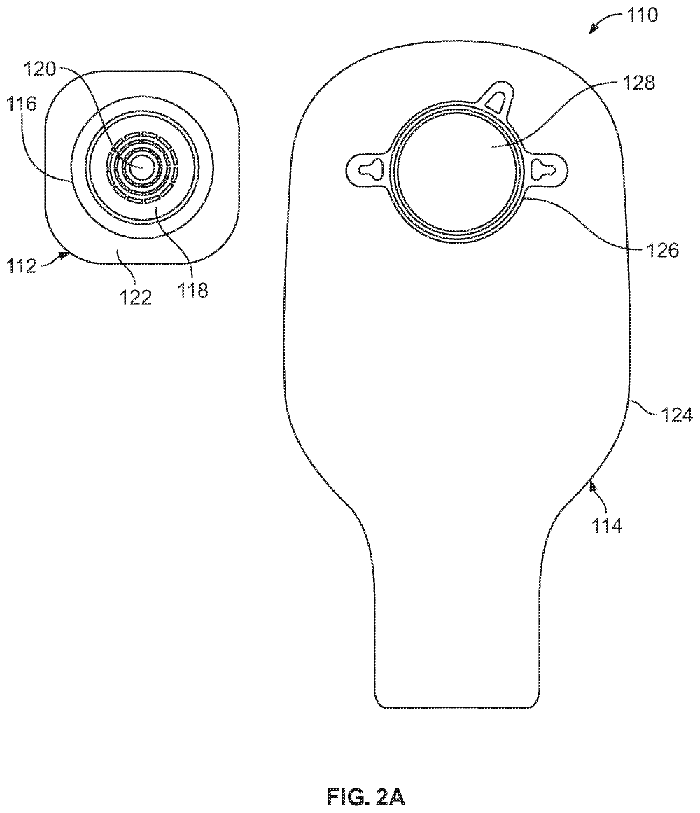

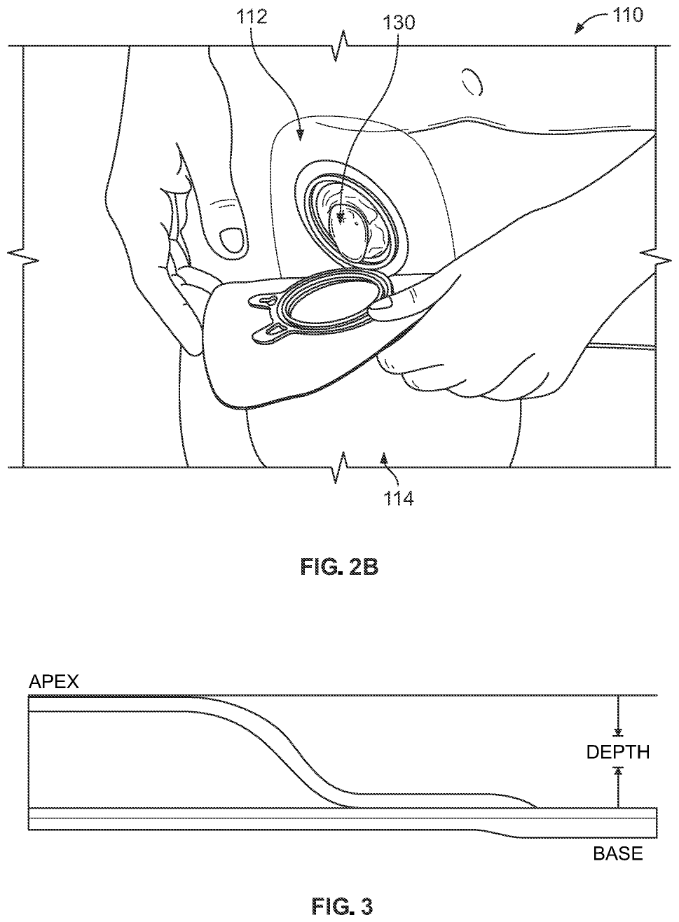

FIG. 2A illustrates an ostomy two-piece pouch system 110. According to example embodiments shown schematically in FIG. 2A, the ostomy system 110 can generally include an ostomy barrier appliance 112 and an ostomy bag 114. The ostomy barrier appliance 112 can include a barrier coupling member 116, a skin barrier 118, an inlet opening 120, and a tape 122. The ostomy barrier appliance 112 can be attached to a user using the tape 122. The ostomy bag 114 can include a drainable pouch 124, an ostomy barrier coupling member 126, and an ostomy bag opening 128. The ostomy bag 114 can be attached to the ostomy barrier appliance 112 by connecting the ostomy barrier coupling member 126 and barrier coupling member 116. The ostomy bag 114 can receive bodily waste through the opening 128 and hold the bodily waste in the drainable pouch 124.

According to example embodiments, the ostomy barrier appliance 112 can include a convex insert 150 (see FIGS. 10-13) that can be attached to the barrier coupling member 116 and abut the skin barrier 118. According to example embodiments, the skin barrier 118 can include a convex skin barrier (FIG. 4).

According to example embodiments, a convex insert may be attached to the barrier coupling member 116. The convex insert 18 can be attached to a backing layer of the tape 122 to provide convexity to the skin barrier 118. In other embodiments, the convex insert may be attached to a pouch-side surface of the skin barrier 118.

FIG. 2B illustrates ostomy pouch system 110 mounted to a user. According to example embodiments shown in FIG. 2B (with corresponding reference to the features of pouch system 110 of FIG. 2A), the ostomy barrier appliance 112 can be attached to a user with a stoma 130 surrounded by the inlet opening 120. The ostomy barrier coupling member 126 can attach to barrier coupling member 116 and receive the stoma through the opening 128 for receiving bodily waste into the drainable pouch 124. In other embodiments, the ostomy pouch may be configured as a one-piece ostomy pouch system including a skin barrier attached to a body side of the pouch and the patient attachment device may be a 1-piece pouch with an attached barrier.

The characteristics of convexity of the skin barrier 118 can include depth, compressibility, flexibility, tension location, and slope. See, McNichol, L., Cobb, T, Depaifve, Y, Quigley, M., Smitka, K., & Gray, M., Characteristics of Convex Skin Barriers and Clinical Application: Results of an International Consensus Panel, J Wound Ostomy Continence Nurs., (2021) 48(6), 524-532, Abstract. According to such teachings, the depth of a convex skin barrier is defined as a distance from the apex of the dome to the base of the skin barrier. Id, at pg. 526. The depth D can be measured as a magnitude of the convexity from the base applied against the peristomal skin to the highest (or outermost/distal) point of the skin barrier as shown in FIG. 3. Id. Individual user's peristomal condition and topography, such as depths of creases and folds around the stoma, should be carefully considered when determining a depth of a convex skin barrier to provide an optimal seal around the peristomal skin. Id.

The compressibility of a convex skin barrier is commonly defined as a capacity of the convex dome to be displaced or flattened as illustrated in FIG. 4. Id, at pg. 528. The compressibility may be measured as a force required to displace or flatten the dome portion of a convex skin barrier by a predetermined distance. A relatively easily compressible soft convex barrier may conform better to users with postoperative edema and/or a relatively firm abdominal. Id. A relatively less compressible firm convex barrier may apply more pressure on the peristomal skin to provide support needed for users with a relatively soft abdominal tone and/or creases around the stoma. Id.

The flexibility of a convex skin barrier is commonly defined as how easily the convex skin barrier can bend, as illustrated in FIG. 5. Id, at pg. 529. The flexibility is an important characteristic to consider when a skin barrier needs to bend to conform to abdominal contours. Id. A relatively more flexible convex skin barrier may work well for users with multiple creases around stoma due to loose skin. Id.

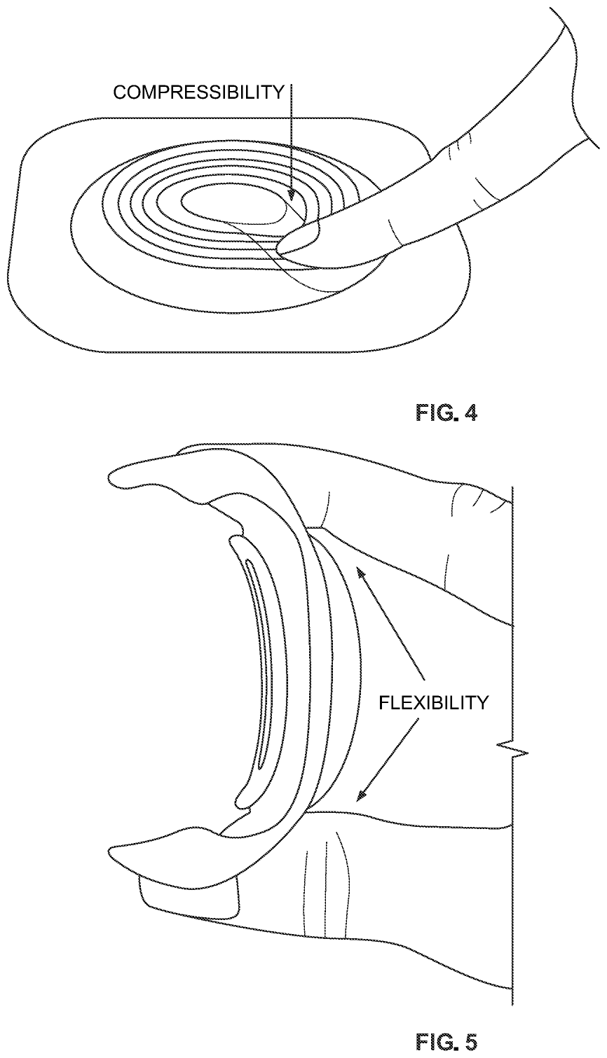

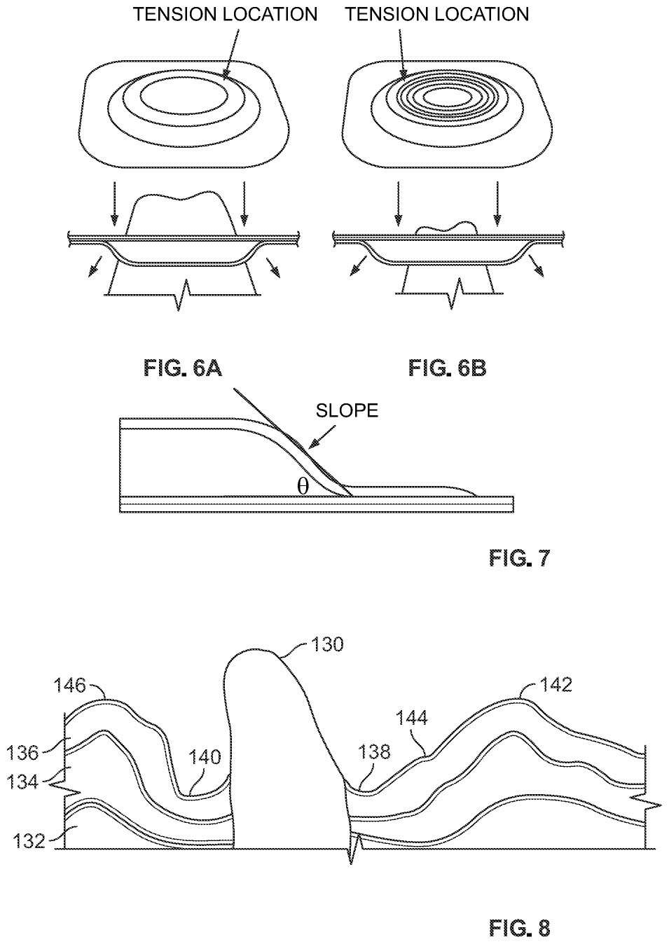

The tension location of a convex skin barrier is commonly identified as the position in which a convex dome exerts downward and outward forces on the peristomal topography, as illustrated in FIGS. 6A and 6B. Id, at pg. 530. A convex skin barrier configured to apply a tension close to a stoma may provide a consistent and reliable seal around the stoma that is flush to the skin or retraced below the skin. Id. For users with creases and folds around the stoma, a convex skin barrier configured to apply a tension away from the stoma may help flatten the peristomal skin to provide a good seal. Id.

The slope of a convex skin barrier is commonly understood as being an angle from the base of the dome to a periphery of the apex of the dome, as illustrated in FIG. 7. Id, at pg. 53. Creases and folds around the stoma can compromise a seal between a skin barrier and the skin. Adjusting the slope of a convex skin barrier according to user's peristomal topography can improve the seal. For example, a convex skin barrier with a relatively small slope and wider plateau may help flatten the peristomal skin creases and folds to achieve a good seal. Id.

Customizing and adjusting the depth, compressibility, flexibility, tension location, and/or slope of a convex skin barrier according to user's peristomal topography can provide an optimal seal around the stoma. The present disclosure provides an ostomy barrier appliance including a convexity adjusting device configured to automatically adjust one or more of the convexity characteristics of a skin barrier according to various embodiments. The convexity adjusting device may be configured as a self-activating device, which may be automatically adjusted and formed according to user's peristomal topography as the ostomy barrier appliance is applied to the user.

FIG. 1 shows a method 10 according to representative embodiments presented herein. According to example embodiments shown schematically in FIG. 1, the method 10 can comprise generating a digital representation of a scanned stoma and peristomal topography. The method may be applied by way of a computing device such as a computing terminal or data processing server.

For example, in step 12, the computing device can obtain a 3D scan of a stoma and peristomal area (FIG. 8). In particular, the computing device can facilitate taking or obtaining a 3D scan of a stomal and/or peristomal area of a person that has had a surgically-created intestinal (e.g. colostomy or ileostomy) tract diversion that creates an abdominal opening for waste elimination. Such scan can be taken by utilizing any number of 3D scanning technologies including utilization of a structured visible light 3D scanner for detecting the three-dimensional shape of an object using projected light patterns and a camera system. Alternate scanning technologies can also be utilized including without limitation optical, acoustic, computed tomography (CT), Magnetic Resonance Imaging (MRI), infrared, Ultrasound, laser scanning, thermal, X-Ray, laser imaging detection and ranging (LIDAR) and other to-be-developed technologies without limitation.

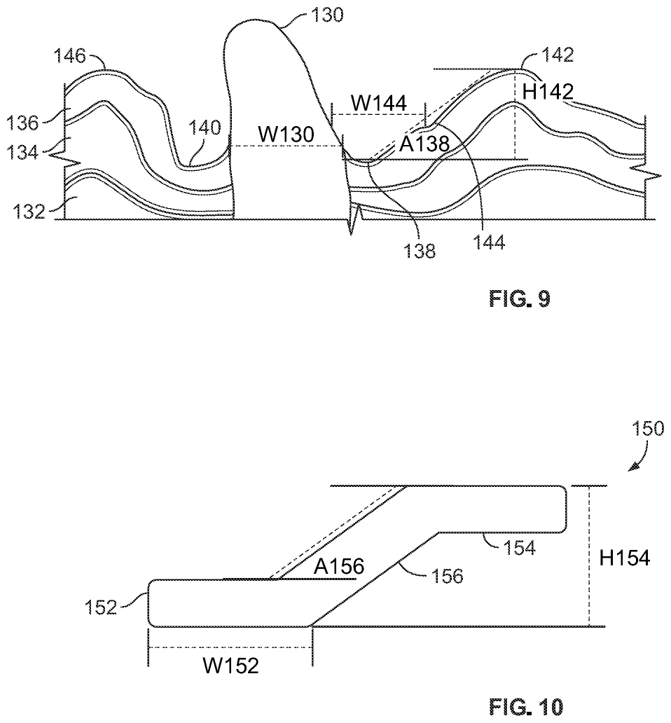

In step 14, the computing device can map the 3D scan of the stoma and peristomal area (FIG. 9). For example, upon scanning the stomal area, the size, shape, topography, location/orientation of the stomal and peristomal surfaces, convexity characteristics, and features can be mapped as data points in space. The data points can comprise a set establishing a point cloud that can be representative of the 3D shape of the stoma and/or peristomal surfaces, where each point position has a set of Cartesian coordinates (X, Y, Z). The computing device, for example, can utilize algorithms programmed to identify a stoma and peristomal surfaces. The algorithms can include artificial intelligence (AI), neural networks, and machine learning models trained to identify a stoma, peristomal surfaces, and convexity characteristics. For example, the algorithm can identify the stoma based on Hounsfield units in a CT or X-Ray scan or proton relaxation times in a magnetic resonance imaging (MRI) scan. Similarly, the skin layer, fat layer, and muscle layer may be identified.

In step 16, the computing device can convert the 3D map to a digital representation. According to example embodiments, the point cloud can be converted 16 into a solid body model which can be representative of the scanned stoma/peristomal area. Such conversion can be undertaken by surface reconstruction utilizing 3D modeling software and techniques, including polygon or triangle mesh models or non-uniform rational basis spline (NURBS) surface modeling. From the subject disclosure it will be understood that the solid body model can have multiple uses relative the development and fabrication of ostomy appliances.

In step 20, the computing device can be used to generate a 3D print of the digital representation. For example, according to example embodiments, a physical 3D representation of the solid body model can be created from the solid body model using 3D printing technology or molding through the use of injection molding or cast molding utilizing a mold filled with a pliable mold material. Where 3D printing is used, material such as plastics, resins, or powders can be joined or solidified together and deposited in successive layers which can account for general surface topography including changes in elevation/depth, uneven surfaces and spaces/openings.

In step 22, the computing device can be used to test the 3D representation. According to example embodiments, the physical 3D representations can be used to assess performance of ostomy concepts and appliances. For example, the 3D representations can be utilized in benchtop test method formats to determine whether particular skin barriers or support rings will adapt and conform to stomal and peristomal surfaces represented in the 3D representation. More particularly, such evaluations can comprise applying a prototype embodying a particular ostomy concept onto at least a portion of the physical three-dimensional representation. It will be appreciated that such utilization can eliminate the need for costly clinical evaluations. It will also be appreciated that such representations can be particularly useful in facilitating the design of ostomy appliances to fit challenging peristomal topographies and be designated as new “characteristics of convexity.” Such data and representations can further be useful to provide guidance to clinicians as to the adaptability of a particular product based on its ability to fit challenging stomal areas.

In step 30, the computing device can be used to fabricate an ostomy appliance such as the convex insert 150 (FIG. 12) from the solid body model, including finished customized ostomy appliances having barrier and/or ring features which can conform to the three-dimensional representation of such model. Said customized ostomy appliances can be fabricated to have a reciprocal shapes, sizes and configurations which closely conform to the surface features, sizes and relative position of an individual's stomal and peristomal characteristics.

FIG. 1 references that the solid body model can have additional uses. For example, in step 40, the set of data points can be stored by a computing device with data points from scans of other persons' stoma and peristomal areas. From such data, stomal characteristics that are prevalent or predominant within a population can be identified by a computing device. For example, data points, point clouds and/or solid body models can be used by a computing device to create set(s) of pre-defined stomal features from a population of data collected from a plurality of scans.

In step 42, the computing device can provide access to the digital representation. According to example embodiments, data regarding predominant stomal characteristics can be made available to persons in need of an ostomy appliance. According to such embodiments, the identified stomal characteristics can be selectable by the person and such characteristics can correspond to an ostomy appliance designed to accommodate such characteristics.

In step 44, the computing device can provide a digital rending of the digital representation. Embodiments presented herein can further include electronically providing digital renderings of an ostomy appliance configured for particular identified characteristics and electronically displaying such renderings together with image data of a user's stomal area. For example, a user interface such as a software program or application (app) can be provided which can help guide the user's selection of an appropriate ostomy appliance that can accommodate the user's corresponding features of their stomal and peristomal area. Such program or application can be configured for interfacing with a digital camera whereby the user can capture or record image data of their stomal region. According to example embodiments, a digital rendering of an ostomy appliance having features for accommodating more predominant stomal characteristics can be displayed over the image output of the user's stomal region so that the user can “virtually try-on” and/or visualize how particular ostomy appliances would fit on and around their stomal area. It will be appreciated that such capabilities can facilitate a user to gauge size, shape and fit or a particular ostomy appliance.

FIGS. 8, 9, and 11 representatively show a cross-section view of a portion of a user's abdomen with a stoma 130. As shown representatively in FIG. 3, the user's abdomen can include the stoma 130, a muscle layer 132, a fat layer 134, and a skin layer 136. The peristomal topography shown in FIGS. 8, 9 and 11 can include a first valley 138, a second valley 140, a first peak 142, a second peak 146, and a middle point 144 between a bottom point of first valley 138 and first peak 142.

FIG. 8 shows the user's abdomen with the stoma 130 protruding out of the abdomen. According to example embodiments shown schematically in FIG. 8, the image can be a 2D or 3D image acquired using various scanning technologies.

FIG. 9 shows annotations relative the peristomal area of FIG. 8. According to example embodiments shown schematically in FIG. 9, the peristomal area can include topographical dimensions including a width W130, an angle A138, a height W142, and a width W144. Width W130 can be a width of the stoma at its widest point or at the point where the stoma protrudes from the skin layer 136. Width W130 can be used to calculate the circumference of the stoma 130 for sizing the opening of the convex insert (FIG. 12). Angle A138 can be a space measured in degrees between a first imaginary line segment horizontally bisecting a bottom point of the first valley and a second imaginary line segment extending from the bottom point of the first valley 138 to first peak 142. Width W144 can be a measurement taken from a point in the stoma 130 that horizontally aligns to a point in the middle section 144. The width W144 can be used to calculate a tension location. Height H142 can be a measurement taken from the bottom point of first valley 138 to the first peak 142. The height H142 can be a convexity depth.

FIG. 10 is a cross-sectional illustration of a portion of a convex insert 150. According to example embodiments shown in FIG. 10, the convex insert 150 can include an inner flange 152, an outer flange 154, and a middle portion 156. The inner flange 152 can have a width W152. The convexity insert can have a height H154 from a bottom of inner flange 152 and a top of outer flange 154. The middle portion 156 can extend between inner and outer flanges 152, 154 and slope at an angle A156 relative an imaginary horizontal axis.

According to example embodiments shown in FIG. 10 (with corresponding reference to FIG. 9), width W152 can be equal to width W144, angle A156 can be equal to angle A138, and height H154 can be equal to height H142. Therefore, the convex insert 150 can be designed to fit the stoma 130 and peristomal topography. In an embodiment, the convex insert 150 can be symmetrical and designed to surround the stoma 130 and peristomal topography based on multiple measurements taken around the stoma 130 and determining a width, height, and angle that best fits the entire stoma 130 and peristomal topography. For example, one of the multiple measurements can include a measurement of a width, height, and angle based on the second valley 140 and the second peak 146. In another embodiment, the convex insert 150 can be asymmetrical and be designed based on multiple widths, heights, and angles measured around the stoma 130 and peristomal topography. For example, a side of the asymmetrical convex insert 150 can be designed based on a measurement of a width, height, and angle of the second valley 140 and the second peak 146. In an embodiment, the width W152 can be adjusted based on the width W130 so that the opening of the convex insert is equal to or greater than W130.

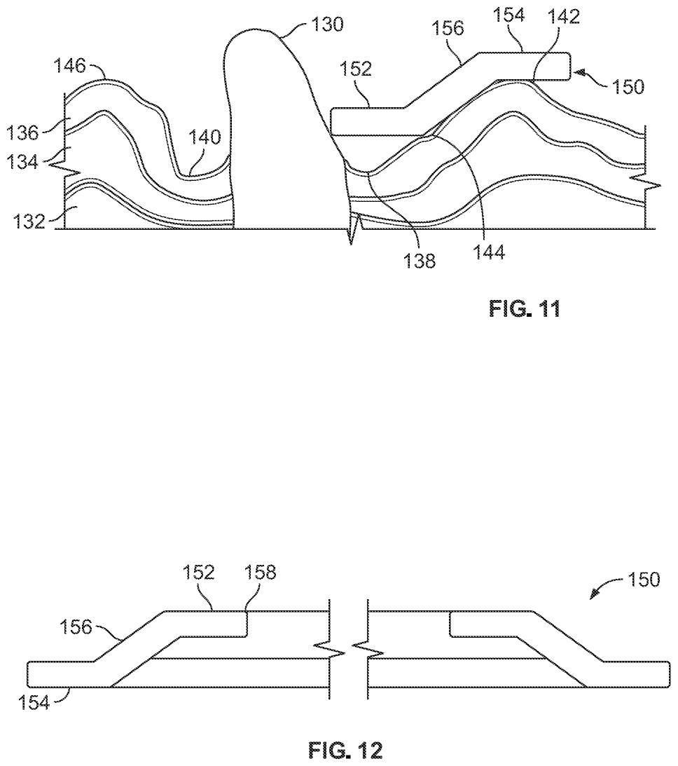

FIG. 11 shows the stoma 130 with a portion of the convex insert 150. According to example embodiments shown in FIG. 11, the convex insert 150 can surround the stoma 130 and provide a better fit for the user. The inner flange 152 can abut the stoma 130, the middle portion 156 can abut the sloping skin layer 136, and the outer layer 154 can abut the peak 142.

FIG. 12 is a cross-section illustration of the convex insert 150. The convex insert 150 can include an opening 158 for surrounding a stoma 130. The opening 158 can be determined based on the Width W130.

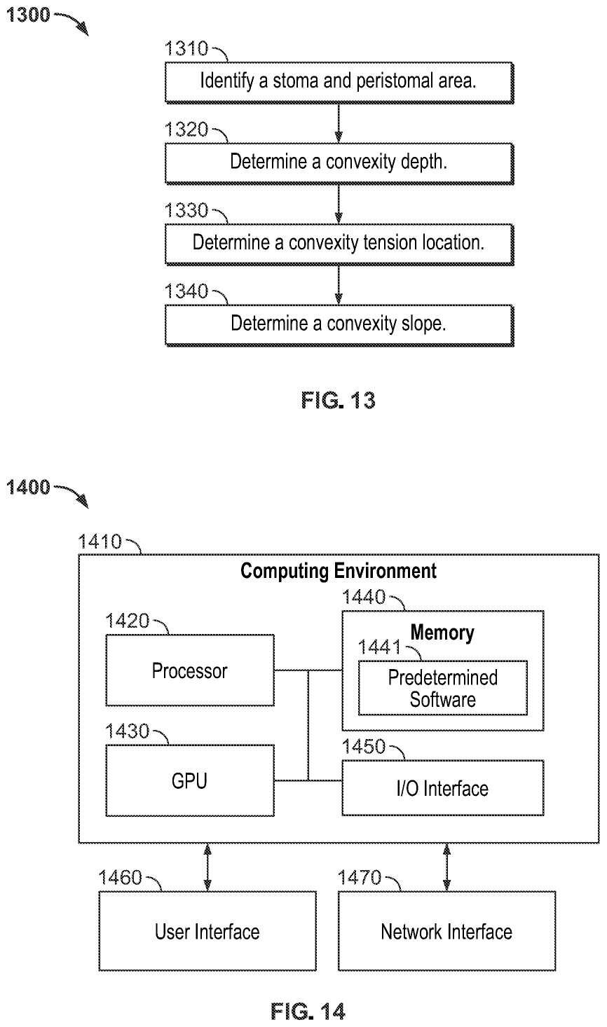

FIG. 13 shows a flow chart illustrating a representative method 1300 for mapping a stoma and peristomal area in an image. The method may be applied to and/or performed by a computing device such as a mobile device, personal computer or server for the fabrication of a custom-fabricated physical ostomy appliance, a physical three-dimension representation of a stomal area for evaluating ostomy appliances and/or for the generation of data to identify predominant stomal and peristomal characteristics for virtual display to a user. The method 1300 can identify the convexity characteristics of the stoma 130 of the type described in connection with FIG. 8 herein, including the depth, tension location, and slope.

In step 1310, the computing device can identify a stoma and peristomal area. For example, the computing device can identify the stoma 130, the muscle layer 132, the fat layer 134, the skin layer 136. The computing device can then identify the topographical features such as protrusions, recesses and sloping surfaces, including those referred to in connection with embodiments described herein (i.e., first valley 138, second valley 140, the first peak 142, the middle section 144, and the second peak 146). According to exemplary embodiments, the computing device, can identify these elements using AI, neural networks, and machine learning models trained to identify or recognize a stoma, peristomal surfaces, and convexity characteristics. For example, the algorithm can identify the stoma based on Hounsfield units in a CT scan or proton relaxation times in an MRI scan. Similarly, the skin layer, fat layer, and muscle layer may be identified based upon common visual characteristics, densities, or compositions.

In step 1320, the computing device can determine a convexity depth. For example, the computing device can determine a height H142 based on the first valley 138 and the peak 142 relative to the stoma 130. The computing device can measure the distance from the outer surface of the skin layer 136 at the first valley 138 to the outer surface of the skin layer 136 at the peak 142. In another example, the convexity depth can be calculated by measuring from the first peak 142 to a “trough” section like the second valley 140.

In step 1330, the computing device can determine a tension location for a convex insert. For example, the computing device can determine the width W144 based on the middle section 144. The computing device can identify the middle section 144 as a point where a tension location can be located. The computing device can measure the width W144 based on the distance between the stoma 130 and the middle section 144. In another example, the tension location can be determined based on measuring from the stoma 130 to an area where the slope starts to increase above 0 (going from flat to raised).

In step 1340, the computing device can determine a convexity slope. For example, the computing device can determine the angle A138 based on the first valley 138, the first peak 142, and an outer edge of the skin layer 136. The computing device can determine the angle A138 based on the slope the outer surface of the skin layer 136 from the first valley 138 to the first peak 142. In another example, the convexity slope can be determined based on measuring from the stoma 130 to an area where the slope starts to increase above 0 (going from flat to raised). In another example, the convexity slope can be determined based on an average slope calculated by taking the distance H142 and dividing it by the angle A138. A specific slope can also be used and it could be calculated based on the same rise/run formula using sub-sections of a cross-section of the image.

FIG. 14 shows a computing system 1400 with a computing environment 1410 coupled with a user interface 1460 and network interface 1470 according to an embodiment. The computing environment 1410 may be part of a data processing server, personal computer, mobile terminal or handheld device. The computing environment 1410 can include a processor 1420, graphical processing units 1430, memory 1440, and I/O interface 1450.

The processor 1420 typically controls overall operations of the computing environment 1410, such as the operations associated with the display, data acquisition, data communications, and image processing. The processor 1420 may include one or more processors to execute instructions to perform all or some of the steps in the above-described methods. Moreover, the processor 1420 may include one or more modules that facilitate the interaction between the processor 1420 and other components. The processor may be a Central Processing Unit (CPU), a microprocessor, a single chip machine, or the like.

The memory 1440 is configured to store various types of data to support the operation of the computing environment 1410. Memory 1440 may include predetermined software 1441. Examples of such data comprise instructions for any applications or methods operated on the computing environment 1410, video datasets, 2D and 3D image data and image scans, etc. The memory 1440 may be implemented by using any type of volatile or non-volatile memory devices, or a combination thereof, such as a static random-access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The I/O interface 1450 provides an interface between the processor 1420 and peripheral interface modules, such as a keyboard, a click wheel, buttons, a touch screen, and the like. The buttons may include but are not limited to, a home button, a start scan button, and a stop scan button. The I/O interface 1450 can be coupled with an encoder and decoder.

Network Interface 1470 provides communication between the processing unit and an external device. The communication can be done through, for example, WIFI or BLUETOOTH hardware and protocols. The Network Interface 1470 may communicate with a mobile network that connects to the internet and webservers.

User interface 1460 may be a mobile terminal or an electronic display.

In some embodiments, there is also provided a non-transitory computer-readable storage medium comprising a plurality of programs, such as comprised in the memory 1440, executable by the processor 1420 in the computing environment 1410, for performing the above-described methods. For example, the non-transitory computer-readable storage medium may be a ROM, a RAM, a CD-ROM, a magnetic tape, a floppy disc, an optical data storage device or the like.

The non-transitory computer-readable storage medium has stored therein a plurality of programs for execution by a computing device having one or more processors, where the plurality of programs when executed by the one or more processors, cause the computing device to perform the above-described method for motion prediction.

In some embodiments, the computing environment 1410 may be implemented with one or more application-specific integrated circuits (ASICs), digital signal processors (DSPs), digital signal processing devices (DSPDs), programmable logic devices (PLDs), field-programmable gate arrays (FPGAs), graphical processing units (GPUs), controllers, micro-controllers, microprocessors, or other electronic components, for performing the above methods.

It is understood that this terminology is non-limiting in nature. In addition, it is understood that one or more various features of an embodiment above may be used in, combined with, or replace other features of a different embodiment described herein.

All patents referred to herein, are hereby incorporated herein in their entirety, by reference, whether or not specifically indicated as such within the text of this disclosure.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A method comprising:
taking a three-dimensional scan of a person's stoma and peristomal areas to generate a set of data points in space which correspond to points along physical external surfaces of the stoma and peristomal areas;
mapping the set of data points into a three-dimensional point cloud, the data points of the point cloud reflecting relative sizes, shapes, convexity characteristics, and topographies of features along external surfaces of the stoma and peristomal areas;
converting the point cloud into a digital three-dimensional solid body model being visually representative of the person's stoma and peristomal areas;
creating a physical three-dimensional representation of the digital three-dimensional solid body model, the physical three-dimensional representation being representative of the person's stoma and peristomal areas; and
using the physical three-dimensional representation for evaluating concepts for implementation in ostomy appliances.

2. The method of claim 1, further comprising:
providing a scanner for the taking of the three-dimensional scan of a person's stoma and peristomal areas, the scanner comprising at least one from a group consisting of a visual light scanner, a scanner utilizing wavelengths of non-visible light, an infrared scanner and a scanner utilizing three-dimensional laser scanning incorporating LIDAR.

3. The method of claim 1, further comprising:
utilizing digital surface reconstruction for converting the point cloud into a digital three-dimensional solid body model.

4. The method of claim 1, wherein the physical three-dimensional representation is created by a three-dimensional printer.

5. The method of claim 1, wherein the physical three-dimensional representation is created by molding through the use of a mold filled with a pliable mold material.

6. The method of claim 1, wherein the evaluating comprises applying a prototype embodying the concept onto at least a portion of the physical three-dimensional representation.

7. The method of claim 1, wherein the topographies of features along the external surfaces of the person's stoma and peristomal areas are reproduced on the physical three-dimensional representation.

8. A method comprising:
taking a three-dimensional scan of a person's stoma and peristomal areas to generate a set of data points in space which correspond to points along physical external surfaces of the stoma and peristomal areas;
mapping the set of data points into a three-dimensional point cloud, the data points of the point cloud reflecting relative sizes, shapes, convexity characteristics, and topographies of features along external surfaces of the stoma and peristomal areas;
converting the point cloud into a digital three-dimensional solid body model being visually representative of the person's stoma and peristomal areas;
electronically storing the set of data points with data points from scans of other persons' stoma and peristomal areas to identify stomal characteristics that are prevalent within a population; and
providing an ostomy appliance to accommodate a stomal region having the characteristics.

9. The method of claim 8, further comprising:
providing a three-dimensional visual light scanner for the taking of the three-dimensional scan of a person's stoma and peristomal areas.

10. The method of claim 8, further comprising:
utilizing digital surface reconstruction for converting the point cloud into a digital three-dimensional solid body model.

11. The method of claim 8, further comprising:
making the stomal characteristics selectable via a user interface.

12. The method of claim 8, further comprising:
visually presenting a digital rendering of the ostomy appliance with image data captured by a digital camera, the image data showing a user's stomal region.

13. The method of claim 8, wherein the ostomy appliance comprises at least one of an ostomy skin barrier and a stoma support ring.

14. The method of claim 8, wherein the ostomy appliance comprises a convex insert with a convexity depth.

15. The method of claim 8, wherein the ostomy appliance comprises a convex insert with a tension location.

16. The method of claim 8, wherein the ostomy appliance comprises a convex insert with a convexity slope.

17. A method comprising:
taking a three-dimensional scan of a person's stoma and peristomal areas to generate a set of data points in space which correspond to points along physical external surfaces of the stoma and peristomal areas;
mapping the set of data points into a three-dimensional point cloud, the data points of the point cloud reflecting relative sizes, shapes, convexity characteristics, and topographies of features along external surfaces of the stoma and peristomal areas;
converting the point cloud into a digital three-dimensional solid body model being visually representative of the person's stoma and peristomal areas;
electronically storing the set of data points with data points from scans of other persons' stoma and peristomal areas to identify stomal characteristics that are prevalent within a population; and
making the stomal characteristics selectable via a user interface, the selectable stoma characteristics corresponding to an ostomy appliance designed to accommodate the characteristics.

18. The method of claim 17, further comprising:
visually presenting a digital rendering of the ostomy appliance with image data captured by a digital camera, the image data showing a user's stomal region.

19. The method of claim 17, further comprising:
providing an ostomy appliance to accommodate a stomal region having the characteristics.

20. The method of claim 19, wherein the ostomy appliance comprises at least one of an ostomy skin barrier and a stoma support ring.
